(19) United States
(12) Patent Application Publication
(10) Pub. No.: US 2025/0325258 A1
(43) Pub. Date: Oct. 23, 2025

(54) Surgical Instrument and Methods of Tissue Repair

(71) Applicant: Arthrex, Inc., Naples, FL (US)

(72) Inventors: Thomas DOONEY, JR., Naples, FL (US); Matthew R. HERRINGTON, Naples, FL (US); Justina HEIDENTHAL, Naples, FL (US); Orr LIMPISVASTI, Santa Monica, CA (US); Joshua S. DINES, Southampton, NY (US)

(73) Assignee: Arthrex, Inc., Naples, FL (US)

(21) Appl. No.: 19/050,278

(22) Filed: Feb. 11, 2025

Related U.S. Application Data
(60) Provisional application No. 63/636,903, filed on Apr. 22, 2024.

(51) Int. Cl. A61B 17/04; A61B 17/00; A61B 17/29

(52) U.S. Cl. CPC A61B 17/0401; A61B 17/29; A61B 2017/0046; A61B 2017/0409; A61B 2017/0464; A61B 2017/2944

(57) ABSTRACT

Instruments and methods for grasping tissue and passing suture and/or fixation devices in tissue repairs and other surgical procedures are disclosed.

### BACKGROUND

The disclosure relates to the field of surgery and, more specifically, to surgical instruments for suturing tissue and associated methods of tissue repairs.

### SUMMARY

Surgical instruments and methods of tissue repairs are disclosed. A surgical instrument can have a jaw assembly with one or more surface features protruding from a surface of one of the jaws of the jaw assembly. A surface feature can be provided directly below the location of a needle pass to identify the exact location of the needle and/or suture pass. A surface feature can identify the position of the instrument on bone when grasping soft tissue and accurately place a fixation device, such as suture anchor, to be passed through it. A surface feature can be also adjusted to go proximal or distal of a needle pass, depending on the passing application. A surgical instrument can be a tissue grasper. A surgical instrument can be a suture passer. A surgical instrument can be a combined tissue grasper/suture passer.

Methods of passing suture and/or grasping tissue are also disclosed. In an embodiment, a surgical instrument is provided with one or more surface features on one of its jaws, to allow identification of and feel a location between the cartilage and osseous footprint, for precise suture passing and/or anchor placement.

### BRIEF DESCRIPTION OF THE DRAWINGS

FIG. 1 illustrates a surgical instrument.

FIGS. 2-5 illustrate a method of tissue repair with the surgical instrument of FIG. 1.

FIG. 6 illustrates another surgical instrument.

FIGS. 7-12 illustrate a method of tissue repair with the surgical instrument of FIG. 6.

FIG. 13 illustrates another surgical instrument.

FIG. 14 is an enlarged top view of a modular bump and associated track of the surgical instrument of FIG. 13.

FIG. 15 illustrates another surgical instrument.

### DETAILED DESCRIPTION

The disclosure provides surgical instruments and methods for securing a first tissue to a second tissue (for example, soft tissue to another tissue) with a surgical instrument configured to grasp tissue and provide exact location of the instrument relative to the anatomy of the surgical site. An instrument can have a jaw assembly with one or more surface features protruding from one of the jaws of the jaw assembly. In an embodiment, a surface feature is located on the bottom of a lower jaw. A surface feature can be a bump, nub, protrusion, protuberance, projection, lump, or any similar structure that provides a feeler to identify different structures with relation to the instrument. A surgical instrument can be a suture passer with location feeler. A surgical instrument can be a tissue grasper/anchor guide with location feeler. A surgical instrument can be a combined suture passer/tissue grasper/anchor guide with location feeler.

In an embodiment, a surface feature can be provided directly below the location of a needle pass to identify the exact location of the needle pass as well as the location of a passing suture. In an embodiment, a surface feature can be provided so that its location can be adjusted to go proximal or distal of the needle, depending on the passing application. A surface feature can also identify the position of the instrument on bone when grasping soft tissue and accurately place a fixation device, such as suture anchor, to be passed through it.

In an embodiment, a surface feature can be provided on one of the jaws of a surgical instrument to help during assessment of tissue reduction by allowing the surgeon to feel the footprint (when there is no visualization for the surgeon). Accuracy of the reduction hinges upon a better understanding of the osseous footprint and geometry during reduction. By adding one or more surface features on a jaw of the instrument, the surgeon can feel and sense the articular margin to help localize ideal suture passing location. The surgeon can feel the transition between smooth cartilage and rough “footprint.” The surface feature(s) can also aid in feeling for anchor bone holes, anchors, and/or sutures coming from the anchors in bone when the surgeon cannot visualize the surgical site. The surface feature(s) act as a guide for passing of fixation devices such as anchors, and/or sutures, and/or any surgical devices through bone, and also for soft tissue reduction, when there is no visualization.

In an embodiment, a surgical instrument can be a suture passer designed to identify its position on bone when passing a needle and/or flexible strands through soft tissue relative to bone. In an embodiment, at least one surface feature can be provided on one of the jaws of the instrument to identify the passing location of the needle pass and/or flexible strand. In an embodiment, the at least one surface feature can be fixed, stationary, non-movable, immobile, and integral with the suture passer. In an embodiment, the at least one surface feature can be non-fixed, movable, and non-integral with the suture passer.

In an embodiment, a surgical instrument can be a tissue grasper designed to identify its position on bone when grasping tissue. The tissue grasper can also allow one or more fixation devices (for example, one or more suture anchors) to be passed through it. Ends of the grasper can be slotted. At least one surface features on one of the jaws of the instrument allows the surgeon to identify and feel the location between the cartilage and the osseous footprint, to properly and accurately place one or more fixation devices. In an embodiment, the at least one surface feature can be fixed, stationary, non-movable, immobile, and integral with the tissue grasper. In an embodiment, the at least one surface feature can be non-fixed, movable, and non-integral with the tissue grasper.

Methods of grasping tissue and/or passing suture and suturing tissue are also disclosed. In an embodiment, a surgical instrument is provided with at least one surface feature on one of its jaws to allow identification and feeling of a location between the cartilage and its footprint, for precise suture passing and/or anchor placement. A surgical instrument can be provided with at least one surface feature on one of its jaws to allow identification and feeling of a transition between two different tissues with different consistencies, for example, a first tissue and a second tissue such as cartilage rim and adjacent bone.

A surgical instrument can include a shaft, an actuating mechanism, and a jaw assembly. A jaw assembly comprises: (i) a first jaw (a first jaw member; a lower jaw; a bottom jaw) and a second jaw (a second jaw member; an upper jaw; a top jaw) movable between a close position and an open position; and (ii) at least one surface feature provided on a surface of one of the first jaw and the second jaw. The at least one surface feature can be a bump, nub, protrusion, protuberance, projection, lump, or combinations thereof, or any similar structure that provides a feeler to identify different structures with relation to the instrument. The at least one surface feature can be a bump protruding from a bottom surface of a lower jaw. The at least one surface feature can be stationary or movable. An actuating mechanism can be coupled to the jaw assembly and configured to move the jaw assembly between the close position and the open position. A surgical instrument can include a handle assembly.

In an embodiment, a surgical instrument comprises: (i) a shaft having a longitudinal axis, a distal end, and a proximal end; (ii) a jaw assembly at a tip of the distal end of the shaft, the jaw assembly including two jaws, wherein one of the two jaws is provided with at least one surface feature located on a surface of the one of the two jaws and on a side opposite the side that contacts the other of the two jaws; and (iii) an actuator configured to move the jaw assembly. At least one surface feature can be stationary. At least one surface feature can be movable. An actuator can be within the shaft and at the distal end of the shaft. A surgical instrument can be a tissue grasper, a suture passer, or combination thereof.

In another embodiment, a surgical instrument comprises: (i) a shaft having a longitudinal axis, a distal end, and a proximal end; (ii) a movable jaw; and (iii) a mounted stationary jaw (stationary member or tip) fixedly mounted to the shaft at the distal end of the shaft, the stationary jaw having one or more surface features located on a surface of the mounted stationary jaw facing away from the movable jaw. At least one surface feature can be stationary. At least one surface feature can be movable. A surgical instrument can also include a handle mechanism configured to move the movable jaw with respect to the mounted stationary jaw. A handle mechanism can further include a locking mechanism for locking the jaw assembly. A surgical instrument can also include an actuator. An actuator can be within the shaft and at the distal end of the shaft. A surgical instrument can also include additional features on at least one of its jaws for grasping and securing soft tissue.

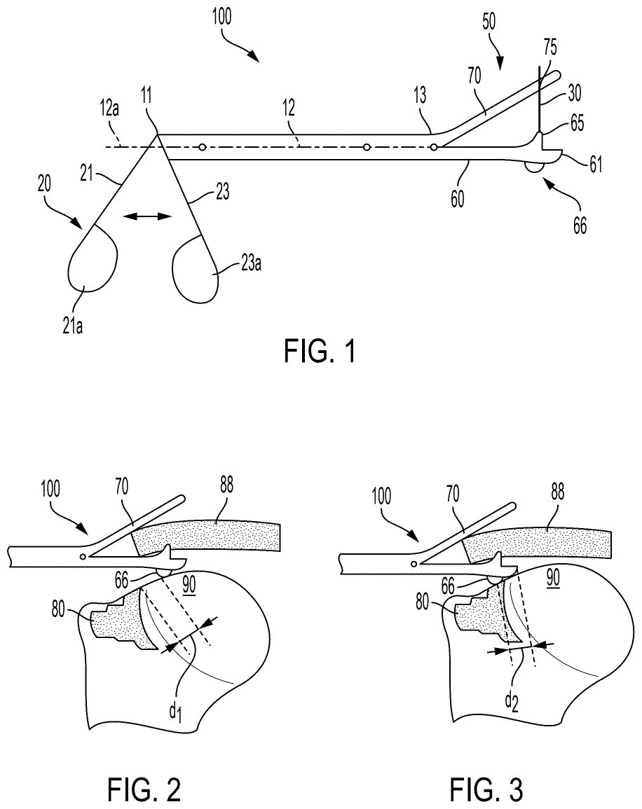

Referring now to the drawings, where like elements are designated by like reference numerals, FIGS. 1-15 illustrate structural elements of surgical instrument 100, 200, 300, 400 (device 100, 200, 300, 400; tissue grasper 100, 200, 300, 400; suture passer 100, 200, 300, 400; grasper with bumps 100, 200, 300, 400; grasper/passer instrument 100, 200, 300, 400; grasper/anchor guide with location feeler 100, 200, 300, 400).

As shown in FIG. 1, instrument 100 includes an elongated tubular member 12 (shaft 12) having a longitudinal axis 12a, a proximal end 11, a distal end 13, and an axial throughbore therein (not shown). Elongated tubular member 12 can be a tube or a narrow-diameter rod of dimensions that permit the tubular member to be introduced through an associated cannula (for example, an 8.25 cannula) in a minimally invasive procedure, such as arthroscopic or other endoscopic procedures, or into a body cavity, such as the abdominal cavity.

Elongated tubular member 12 connects, and extends between, a handle assembly 20 and a tip 50 designed to pierce tissue. Tip 50 is provided with a jaw assembly formed of first and second jaws 60, 70 (lower and upper jaws 60, 70; tines 60, 70; first and second jaw members 60; 70) provided integral with shaft 12. One of the first and second jaws 60, 70 can be a stationary jaw and the other of the first and second jaws 60, 70 can be a movable jaw. In an embodiment, first jaw 60 is a stationary jaw 60 (stationary member 60; static member 60; tip 60; mounted stationary jaw 60) provided at the distal end 13 of the instrument and can be integral with (fixedly mounted to) the tubular member 12 at the distal end 13 of the shaft. In an embodiment, second jaw 70 is a movable jaw 70 (movable member 70) and first jaw 60 is a stationary jaw 60. In an embodiment, both jaws 60, 70 can be movable with respect to each other and the instrument.

In an embodiment, first jaw 60 is a lower jaw provided with a distal point 61, a passage (not shown) for receiving a needle 30, and a distal opening 65 to allow the needle to pass through. Opening 65 is provided at a distal end of tip 50 and communicates with the needle passage. Distal opening 65 allows needle 30 to engage at least one flexible strand 35 (for example, suture 35) to exit in a direction non-parallel to the longitudinal axis of the instrument. Distal point 61 of lower jaw 60 can be rounded. In an embodiment, flexible strand 35 can be positioned on the opening 65 to allow engagement with needle 30.

In an embodiment, second jaw 70 is an upper jaw provided with a distal opening 75 to allow the needle 30 and flexible strand 35 to pass through. Opening 75 can be a through opening that allows a needle and a portion of flexible strand to pass and extend therethrough.

As also shown in FIG. 1, first jaw 60 is provided with at least one surface feature 66 to allow a surgeon to feel an articular margin to help localize ideal needle and suture passing location when there is no visualization of the surgical site. By providing surface feature 66 on a bottom of the jaw 60, the surgeon can “feel” the transition between smooth cartilage and rough “footprint” and, thus, feel the position of the instrument relative to the bone. The surface feature 66 can also aid in feeling for anchor bone holes, anchors, and/or sutures coming from the anchors in bone, as detailed below.

Surface feature 66 can be a bump, nub, protrusion, protuberance, projection, lump, or any similar structure that provides a feeler to identify different structures with relation to the instrument 100. In an embodiment, and as shown in FIG. 1, surface feature 66 is located at the distal end of lower jaw 60 and on surface of the lower jaw opposite the surface where the distal end 65 of the needle passage is located (i.e., at the opposite end of the needle exiting location). Surface feature 66 can have an about semicircular or dome-like configuration, to allow a surgeon to feel the footprint during assessment of reduction, i.e., to feel the articular margin to help localize ideal suture passing location when there is no visualization. In an exemplary-only embodiment, surface feature 66 can be a bump or a nub of about 1 mm to about 2 mm to allow a surgeon to feel the stepoff between the articular margin and the tuberosity in, for example, a rotator cuff repair. One or more surface features 66 can identify and locate a transition between different tissues with various consistencies (different densities) such as cartilage rim and adjacent bone.

Surface feature 66 is located on a surface of the jaw 60 that is opposite an active side of the instrument. For example, surface feature 66 is located on a surface opposite to the surface that interacts with the anatomy. A surface that interacts with the anatomy can include a gripping surface, or a suture passer needle passing surface, etc. In the exemplary embodiment of FIG. 1, surface feature 66 is located about right below distal opening 65 that allows needle 30 to pass through. In an embodiment, surface feature 66 can be offset from the distal opening 65 by a distance of about 0 to about 2 mm, preferably about 0 to about 1 mm.

Surface feature 66 acts as a tactile element in that it allows a surgeon to feel the location/position of the instrument relative to the bone/cartilage/soft tissue when there is no visualization. As the surgeon cannot see the field or surgical site, accurate needle passing and/or fixation device insertion is paramount. The surface feature 66 allows accurate trial reduction prior to needle passage and identification of the osseous footprint location and geometry during tissue reduction.

Handle assembly 20 (grip assembly 20; handle 20) of instrument 100 (illustrated in FIG. 1) is provided at the proximal end 11 of the instrument and includes a pair of cooperating front and rear handles 21, 23 (proximal and distal handles 21, 23), respectively, at the proximal end 11 of shaft 12. The handles 21, 23 are relatively movable to position and reposition the movable jaw 70 between a first position (shown in FIG. 1) and a second position wherein the jaw 70 is moved towards the stationary jaw 60. The handles 21, 23 each have a finger loop 21a, 23a to accept, for example, the thumb and middle finger of the user. By squeezing the handles 21, 23 towards each other, an operating rod (not shown) can be repositioned to urge the jaw 70 into a second position. The closing force on the jaws 60, 70 is determined by the amount of pressure applied through the handles 21, 23. Opening of the jaws 60, 70 can be accomplished by spreading the handles 21, 23 away from each other.

Handle assembly 20 can also include a locking mechanism connected to the operating handles 21, 23 for locking and unlocking the jaw assembly 50 (i.e., for locking and unlocking of the first and second jaws 60, 70, as desired by the user). In an embodiment, the locking mechanism can be a ratchet mechanism (not shown). In an embodiment, a ratchet mechanism can include an additional finger loop, to allow the user to lock and/or unlock the jaw assembly 50 at different positions, including intermediary positions between an open configuration and a close position.

Instrument 100 further includes an actuator (not shown), which is provided within the shaft 12 and at the distal end of the shaft. The actuator is connected to the handles 21, 23 and is designed to move the movable jaw 70 from a first position (for example, open position) to a second position (for example, a close position) as each of the handles 21, 23 is moved from a first position to a second position, and to grasp and secure tissue between the two jaws, as detailed below.

Reference is now made to FIGS. 2-5 which illustrate a sequence of steps of exemplary rotator cuff repair 101 with instrument 100. For simplicity, only the distal end of the jaw assembly 50 at the distal end 13 of the tip of instrument 100 is illustrated.

### FIG. 2

The addition of smooth “bump” 66 directly below the location of the needle pass of the instrument 100 can identify the passing location by feeling the transition between cartilage 80 and the osseous “footprint” 90. The cartilage is smooth and the footprint is rough. Smooth bump 66 can also feel for anchor bone holes, anchors and/or sutures coming from the anchors in bone.

As depicted in FIG. 2, bump 66 feels smooth so the pass is too far medial by a distance d1. Surgeon needs to readjust grasp on soft tissue 88.

### FIG. 3

The bump 66 feels rough so the pass is too far lateral by a distance d2. Surgeon needs to readjust grasp on tissue 88.

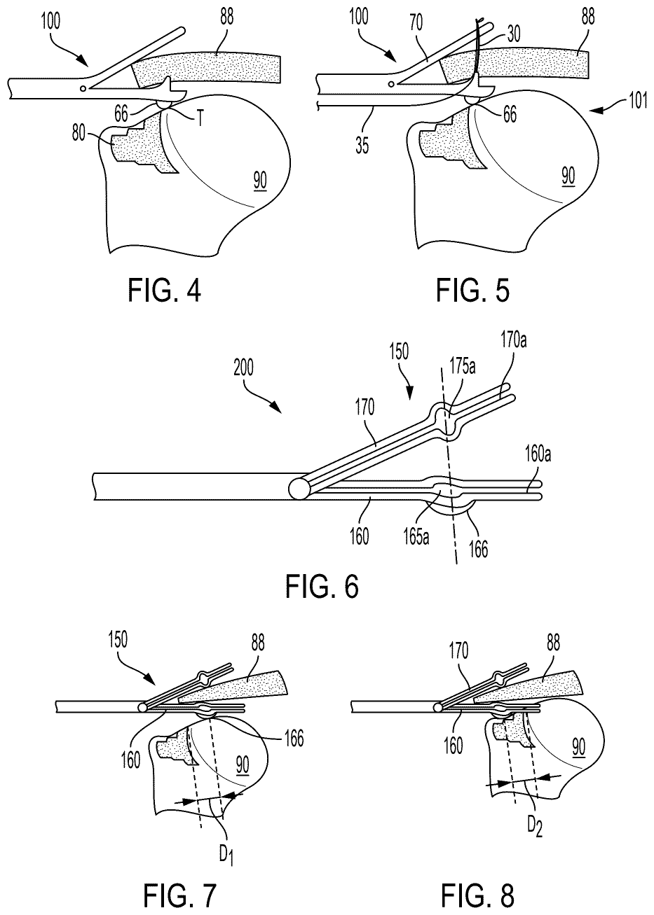

### FIG. 4

The bump 66 feels the location of transition T between smooth and rough, so the pass is in the ideal location.

### FIG. 5

Once the ideal location has been identified at transition point T, jaws 60, 70 can be locked so instrument 100 is secured at the ideal position while grasping tissue 88, and to allow needle 30 to pass through distal opening 65 of jaw 60. Needle 30 can pass through soft tissue 88 and through the jaw 70 with a flexible strand 35, to form a first suture pass as part of repair 101. Additional suture passes can be formed as desired. In additional embodiments, needle 30 can be passed through jaw 60, tissue 88, and jaw 70 to capture a flexible strand and then pass the captured strand back through tissue 88 (when the needle 30 is retracted).

Reference is now made to FIG. 6 which illustrates surgical instrument 200 of the present disclosure. Instrument 200 is about similar to instrument 100 described above, in that it is also designed to securely engage tissue and allow the surgeon to feel the osseous footprint (i.e., the surgeon can feel the articular margin and the transition between smooth cartilage and rough “footprint”). However, instrument 200 is provided with a jaw assembly 150 that includes a pair of slotted jaws 160, 170 (slotted end effectors 160, 170). A surface feature 166 (bump 166) is provided on a surface of one of the slotted jaws 160, 170 to allow a surgeon (user) to identify and feel the location between the cartilage and footprint to properly place a suture anchor, as detailed below.

Slotted openings 165, 175 are through openings which are about reciprocal and similar in size and provided within and through respective first and second jaws 160, 170 (as shown in FIG. 6). Slotted openings 165, 175 can have various configurations to allow one of more fixation devices (such as one or more suture anchors) and/or one or more flexible strands to pass therethrough. Slotted openings 165, 175 can run in a direction about longitudinal to each longitudinal axis of jaws 165, 175 and communicate with a most distal surface 160a, 170a of each of the two jaws.

In the embodiment of FIG. 6, surface feature 166 is provided on a bottom surface of bottom slotted jaws 160 and directly below opening 165a of slotted jaw 160. Opening 165a communicates with reciprocal Openings 165a, 175a allow one or more fixation devices to pass therethrough and through a soft tissue, to aid in a tissue repair wherein a surgeon (user) can identify and feel the location between the cartilage and footprint to properly place the fixation devices, as detailed below. In an embodiment, surface feature 166 can be offset from the opening 165a by a distance of about 0 to about 2 mm, preferably about 0 to about 1 mm. In an embodiment, distal surface 166 can be offset from most distal end of the instrument by about 1 to about 10 mm, more preferably by about 1 to about 5 mm.

Reference is now made to FIGS. 7-12 which illustrate a sequence of steps of exemplary rotator cuff repair 201 (FIG. 12) with surgical instrument 200. For simplicity, only the distal end of the jaw assembly 150 at the distal end 13 of the tip of instrument 200 is illustrated.

### FIG. 7

The addition of smooth “bump” 166 directly below the location of the anchor pass allows precise placement of an anchor to be passed through the jaws 160, 170 of instrument 200, when the surgeon cannot visualize the surgical site. The ends of the grasper are slotted to allow the sutures to freely release as the grasper 200 is removed from the tissue 88. The location of where the anchor is inserted can be viewed and felt with this design. Bump 166 acts as a location feeler and indicator for tissue grasper/anchor guide 200.

The addition of a location bump 166 allows the user to identify, locate, detect, and feel the location T between the cartilage and footprint to properly place a suture anchor.

As depicted in FIG. 7, bump 166 feels smooth so the anchor location is too far medial by a distance D1. Surgeon needs to readjust grasp on soft tissue 88.

### FIG. 8

The bump 166 feels rough so the pass is too far lateral by a distance D2. Surgeon needs to readjust grasp on tissue 88.

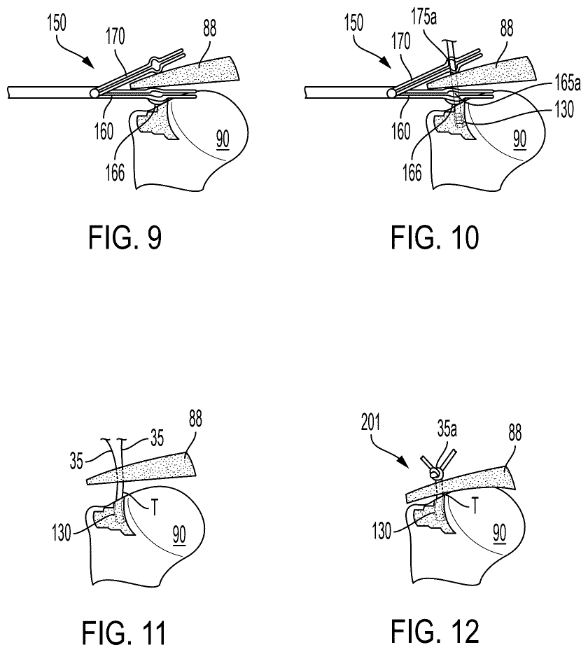

### FIG. 9

The bump 166 feels the location of transition T between smooth and rough, so the anchor location is in the ideal location.

### FIG. 10

Once the ideal location for the anchor has been identified at transition point T, jaws 160, 170 can be locked so instrument 200 is secured at the ideal position, and to allow fixation device 130 (suture anchor 130) to pass through distal opening 175a, 165a. The anchor is inserted through the guide 200 and tendon 88 at the transition point T between cartilage and footprint.

### FIG. 11

The grasper 200 is removed leaving the sutures 35 passed through tissue 88. Anchor 130 is secured within bone 90.

### FIG. 12

The sutures 35 can be tied (to form at least one knot 35a) or fixated to secure tendon 88 to bone 90 as part of repair 201. Additional suture passes can be formed and additional fixation devices can be secured within bone 90 with guide 200, if necessary and as desired.

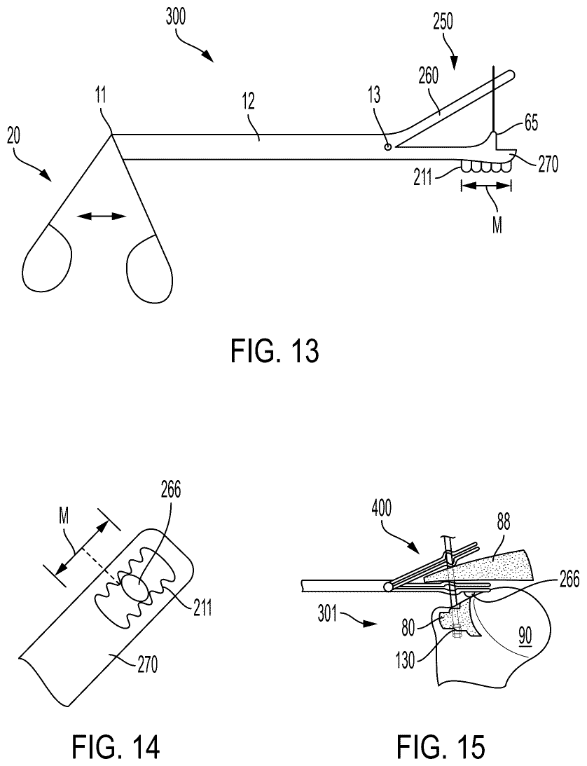

Reference is now made to FIGS. 13 and 14 which illustrate another surgical instrument 300 of the present disclosure. Instrument 300 is about similar to instrument 100 described above, in that it is also designed to securely engage tissue and allow the surgeon to feel the osseous footprint (i.e., the surgeon can feel the articular margin and the transition between smooth cartilage and rough “footprint”) to pass a needle. However, instrument 300 is provided with a jaw assembly 250 that includes a pair of jaws 260, 270, wherein jaw 270 is provided with a movable surface feature 266 (bump 266) provided on a surface of jaw 270 to allow a surgeon (user) to identify and feel the desired location between the cartilage and footprint. As shown in FIGS. 13 and 14, surface feature 266 can travel along distance M relative to needle opening 65. Surface feature 266 can be adjusted to go proximal or distal of the passing needle depending on the passing application. Surface feature 266 can travel along track 211 (or any similar structure such as a razor knife-type device) as the surgeon moves it forward or backward. The surgeon can adjust the position of surface feature 266 on the track intraoperatively, if necessary. Surgical instrument 300 is user configurable in that it includes an adjustable offset.

Surface feature 266 (modular bump 266) can be also part of a grasper such as instrument 400 shown in FIG. 15. As part of repair 301, the user (surgeon) may want to feel and identify the anatomy under soft tissue 88 but insert a fixation device 130 (such as a suture anchor 130, for example) about 5 mm distal to this location. The bump 266 can be adjusted in the operating room or can be permanently offset.

The disclosure provides more accurate trial reduction prior to needle passage. This aspect helps avoid misplacement of the suture relative to the implant (suture anchor) which leads to both malreduction of the tendon and/or type 2 failures. Accuracy of the reduction hinges upon a better understanding of the osseous footprint location and geometry during reduction; instrument 100, 200 of the present disclosure greatly improves this necessary accuracy.

Instrument 100, 200, 300, 400 can also include a rachet to allow for manipulating the tendon/tissue. Instrument 100, 200, 300, 400 can also include one or more friction elements (such as teeth, protuberances, etc.) to the end of the jaw(s) to improve this function. Instrument 100, 200, 300, 400 can also include measurements on the jaw(s); visible measurements on the jaw(s) can allow for measurement of the osseous footprint as well as the tendon width (medial to lateral) for tissue repairs such as rotator cuff repairs. Instrument 100, 200, 300, 400 can also include a pair of jaws (such as jaws 60, 70, 160, 170, 260, 270) that have different lengths. For example, a more rounded and shorter jaw could better grasp thicker tissue and avoid overbiting the tendon (passing the needle close to the musculotendinous junction). Instrument 100, 200 can also include more than two jaws.

Flexible strand 35 (coupler 35; flexible material 35; suture tape 35; suture 35) can be formed of suture, either round and/or flat suture, for example, a suture strand or suture tape. Flexible strand 35 can be an elastic suture.

Fixation device 130 can be an anchor (knotted anchor, knotless anchor, or all-suture anchor), implant, button, screw or any fixation device that confers secure attachment and fixation of soft tissue 88 over bone 90 and that allow sliding of suture within a body of fixation device. The fixation device 130 can be a knotless suture anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein.

Fixation device 130 can be a soft anchor or an “all-suture” anchor. A soft anchor (soft suture anchor or all-suture soft knotless anchor) is provided with a soft anchor sleeve (sheath or tubular member) with two open ends, and at least two flexible shuttling strands extending through the soft anchor sleeve (sheath). The flexible strands may extend through the sleeve in similar or different directions and/or orientations and/or locations. The flexible tubular sleeve with the shuttling strands may be secured into or onto bone, and flexible strands may pass over soft tissue (rotator cuff) and are secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

The surgical instrument and constructs detailed above have applicability to any tissue repair and surgical procedure such as, for example, rotator cuff repairs, Achilles tendon repair, patellar tendon repair, ACL/PCL reconstruction, hip and shoulder reconstruction procedures, and applications for elastic suture used in or with suture anchors.

A method of tissue repair 101, 201, 301 comprises identifying a transition location T between cartilage and osseous footprint with a surgical instrument 100, 200, 300, 400. The step of identifying a transition location T can include feeling a location between the cartilage and the osseous footprint with at least one surface feature 66, 166, 266 provided on a jaw 60, 70, 160, 170, 260, 270 of instrument 100, 200, 300, 400. The surface feature 66, 166, 266 can be stationary and formed integral with the instrument 100, 200, 300, 400. The surface feature 66, 166, 266 can be adjusted in the operating room.

A method of tissue repair 101 comprises inter alia the steps of: (i) identifying a transition location T between cartilage and osseous footprint with a surgical instrument 100, 300; and (ii) conducting at least one suture pass with suture 35 at the transition location T. Identifying the transition location T can be conducted with a surface feature 66, 266 provided on one of jaws 60, 70, 260, 270 of instrument 100, 200. Suture 35 can be passed through soft tissue 88 to be attached to bone 90. The surface feature 66, 266 can be stationary or movable.

A method of tissue repair 201, 301 comprises inter alia the steps of: (i) identifying a transition location T between cartilage and osseous footprint with a surgical instrument 200, 400; and (ii) securing at least one fixation device 130 at the transition location T. The fixation device 130 can be secured within bone 90. The fixation device 130 can be a hard body anchor or a soft body anchor. The fixation device 130 can be a knotted or knotless device. Identifying the transition location T can be conducted with a surface feature 166, 266 provided on one of jaws 160, 170 of instrument 200, 400. The surface feature 166, 266 can be stationary or movable.

A surgical instrument 100, 200, 300, 400 comprises: (i) a shaft 12 having a longitudinal axis 12a, a distal end 13, and a proximal end 11; (ii) a plurality of jaws 60, 70, 160, 170, 260, 270 at the distal end 13 of the shaft 12, at least one of the plurality of jaws being provided with a surface feature 66, 166, 266 to identify a location between cartilage and osseous footprint; and (iii) an actuator configured to move at least one of the plurality of jaws 60, 70, 160, 170, 260, 270. An actuator can be within the shaft 12 and at the distal end of the shaft. A surgical instrument 100, 200, 300, 400 can be one of a tissue grasper, suture passer, and/or tissue grasper/anchor guide/suture passer.

A jaw assembly 50, 150, 250 comprises: (i) a first jaw 60, 160, 260 (a first jaw member; a lower jaw; a bottom jaw) and a second jaw 70, 170, 270 (a second jaw member; an upper jaw; a top jaw) movable between a close position and an open position; and (ii) at least one surface feature 66, 166, 266 provided on a surface of one of the first jaw and the second jaw 60, 70, 160, 170, 260, 270. The at least one surface feature 66, 166, 266 can be a bump, nub, protrusion, protuberance, projection, lump, or combinations thereof, or any similar structure that provides a feeler to identify different structures (different structure densities) with relation to the instrument. The at least one surface feature 66, 166, 266 can be a bump having a dome-like configuration and protruding from a bottom surface of a jaw (opposite to an active side that interacts with the anatomy and can include the gripping surface, suture passer needle passing surface, etc.). The at least one surface feature 66, 166, 266 can be stationary or movable relative to the surgical instrument (and relative to the bottom surface of the jaw). An actuating mechanism can be coupled to the jaw assembly 50, 150, 250 and configured to move the jaw assembly between a close position and an intermediary or open position. A surgical instrument 100, 200, 300, 400 can include a handle assembly 20.

A flexible strand 35 can include a high-strength suture, such as an ultrahigh molecular weight polyethylene (UHMWPE) suture. High strength suture may be a FiberWire® suture sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Flexible strand 35 can be braided or multi-filament suture such as FiberTape® suture tape (as disclosed in U.S. Pat. No. 7,892,256, the disclosure of which is incorporated in its entirety herewith).

Flexible strand 35 can be formed of various flexible materials and strands such as round suture, flat suture, ribbon, or flat tape (for example, suture tape) or combination of suture and tape. Exemplary materials can include suture, silk, cotton, nylon, polypropylene, polyethylene, ultrahigh molecular weight polyethylene (UHMWPE), polyethylene terephthalate (PET), and polyesters and copolymers thereof, or combinations thereof. Flexible strand 35 can be elastic suture or can include elastic material. Flexible strand 35 can consist essentially of elastic material such as elastane. Flexible strand 35 can have cross-sections of various forms and geometries, including round, oval, rectangular, or flat, among others, or combination of such forms and geometries.

Flexible strand 35 can be also formed of a stiff material, or combination of stiff and flexible materials. Various regions, or sections of flexible strand 35 can be coated and/or provided in different colors for easy manipulation during the surgical procedure. Easy identification of suture in situ is advantageous in surgical procedures, particularly during arthroscopic surgeries, endoscopic and laparoscopic procedures.

A surface feature 66, 166, 266 can be provided on a jaw to help during assessment of reduction by allowing the surgeon to feel the footprint. Accuracy of the reduction hinges upon a better understanding of the osseous footprint and geometry during reduction. By adding a surface feature on a jaw surface of the instrument, the surgeon can feel the articular margin to help localize ideal suture passing location.

The surface feature 66, 166, 266 helps a surgeon identify the passing location by allowing the surgeon to “feel” the transition between smooth cartilage and rough “footprint,” for example, in situation when site visualization is unavailable. The surface feature 66, 166, 266 can also aid in feeling for anchor bone holes, anchors, and/or sutures coming from the anchors in bone. A surface feature 66, 166, 266 can be a bump, nub, protrusion, protuberance, projection, lump or any similar structure provides a feeler to identify different structures with relation to the grasper, and the location of these structures. A surface feature 66, 166, 266 on a jaw of the instrument allows the surgeon to identify and feel the location between the cartilage and the footprint, to properly and accurately assess a needle pass and/or to place a fixation device such as a suture anchor.

An instrument is a suture passer with location feeler. More accurate trial reduction prior to needle passage. This will help avoid misplacement of the suture relative to the implant leading to both malreduction of the tendon and/or type 2 failures. Part of the accuracy of the reduction also hinges upon a better understanding of the osseous footprint location and geometry during reduction.

A rachet can be added to allow for manipulating the tendon/tissue. Inclusion of a friction element such as teeth to the end of the jaw would improve this function. Visible measurements on the jaw would allow for measurement of the osseous footprint as well as the tendon width (medial to lateral) for tissue repairs such as rotator cuff repairs. A more rounded and shorter jaw could better grasp thicker tissue and avoid overbiting the tendon (passing the needle close to the musculotendinous junction.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A surgical instrument comprising:
a shaft having a longitudinal axis, a distal end, and a proximal end;
a first jaw and a second jaw, wherein the first jaw and the second jaw are located at the distal end of the shaft, and wherein the first jaw includes at least one surface feature located on a surface of the first jaw that is opposite another surface of the first jaw facing the second jaw; and
a handle assembly configured to move at least one of the first jaw and the second jaw.

2. The surgical instrument of claim 1, wherein the at least one surface feature is a bump, nub, protrusion, protuberance, projection or lump.

3. The surgical instrument of claim 1, wherein the at least one surface feature extends away from the surface of the first jaw.

4. The surgical instrument of claim 1, wherein the at least one surface feature is stationary and integral with the first jaw.

5. The surgical instrument of claim 1, wherein the at least one surface feature is movable relative to the first jaw.

6. The surgical instrument of claim 1, wherein the another surface of the first jaw is a needle passing surface or a tissue grasping surface.

7. The surgical instrument of claim 1, wherein the first jaw is a lower jaw and the at least one surface feature acts as a feeler to identify at least one structure in relation to the surgical instrument.

8. The surgical instrument of claim 7, wherein the at least one structure is cartilage rim or osseous footprint.

9. The surgical instrument of claim 7, wherein the at least one surface feature allows a user to determine and locate a transition between a first tissue with a first density and a second tissue with a second density, wherein the first density is different from the second density.

10. The surgical instrument of claim 1, wherein the at least one surface feature is located about below a needle passage located within the first jaw.

11. The surgical instrument of claim 1, wherein the handle assembly includes a pair of handles and a locking mechanism.

12. The surgical instrument of claim 1, wherein the instrument is a tissue grasper.

13. The surgical instrument of claim 1, wherein the instrument is a suture passer.

14. The surgical instrument of claim 1, wherein the first jaw and the second jaw are slotted to allow passage of at least one fixation device.

15. The surgical instrument of claim 14, wherein the at least one fixation device is a suture anchor.

16. A method of passing at least one flexible strand through soft tissue with a surgical instrument as recited in claim 1 comprising:
identifying a transition between a first tissue and a second tissue; and
passing the at least one flexible strand at the transition between the first tissue and the second tissue.

17. The method of claim 16, wherein the step of identifying the transition between the first tissue and the second tissue comprises moving the at least one surface feature relative to the first jaw and in a forward or backward motion.

18. The method of claim 16, wherein the step of identifying the transition between the first tissue and the second tissue comprises feeling with the instrument a difference between the first tissue and the second tissue and without visualizing the first tissue and the second tissue.

19. The method of claim 18, wherein the first tissue is cartilage rim and the second tissue is bone.

20. The method of claim 16, further comprising passing at least one fixation device through the soft tissue and at the transition between the first tissue and the second tissue.
